(19) United States
(12) Patent Application Publication
(10) Pub. No.: US 2025/0031983 A1
(43) Pub. Date: Jan. 30, 2025

(54) Apparatus or Method for Determining Blood Pressure of a Subject

(71) Applicant: Nanyang Technological University, Singapore (SG)

(72) Inventor: Jun LUO, Singapore (SG)

(21) Appl. No.: 18/764,956

(22) Filed: Jul. 5, 2024

(30) Foreign Application Priority Data
Jul. 5, 2023 (SG) 10202301919X

(51) Int. Cl. A61B 5/021

(52) U.S. Cl. CPC A61B 5/02125

(57) ABSTRACT

An apparatus, for determining blood pressure of a subject is provided. The apparatus comprises at least one processor, at least one memory comprising computer program code, and a radio frequency (RF) wave device configured to detect pulse waveform signals from the subject. The at least one memory and the computer program code are configured to, with the at least one processor, determine an estimation of the blood pressure corresponding to the pulse waveform signals using a blood pressure specific transfer function (BTF) derivation model.

### PRIORITY CLAIM

The present application claims priority to Singapore Patent Application No. 10202301919X, the complete disclosure of which is hereby incorporated by reference.

### TECHNICAL FIELD

The present invention generally relates to an apparatus and a method for estimating blood pressure of a subject.

### BACKGROUND ART

A blood pressure (BP) measurement is significant to the assessment of many dangerous health conditions in subjects. Hypertension (high blood pressure) often develops over many years without any symptoms and could cause heart attack, stroke, chronic heart failure, and kidney disease.

There are various methods of measuring the BP including invasively inserting catheters into arteries which causes discomfort to the subject, and non-invasive approaches which mostly rely on wearable instruments that potentially cause discomfort and yield uncertain accuracy under variable wearing states. An example of a non-invasive BP measurement device is oscillometry, which requires strapping on inflatable cuffs to block blood flow causing discomfort. Furthermore, oscillometry is usually sensitive to cuff type resulting in inconsistent results. Some attempts have been made to reduce discomfort by integrating inflatable cuffs into earbuds and smartwatches, however, these measurements are still sensitive to device-wearing states, hindering reliable BP monitoring. Other methods require multiple sensors to be attached to the body which are dependent on sensor placement on the body and are inconvenient.

Some contactless methods utilize cameras which require strict recording situations such as adequate lighting and the entire view of the subject's face incurring privacy concerns. Other methods leverage radio frequency (RF) technologies which overfits a deep learning (DL) model to map morphological features to the BP. However, the learned mapping between morphological features and the BP may not be causal and lack physiological basis making its validity and reliability questionable. Another disadvantage is these systems require high volumes of training data to cope with variations in morphological features across different subjects and measurement scenarios.

It is an object of at least an embodiment of the invention to provide an apparatus or a method which goes some way toward overcoming these disadvantages or at least provide the public with a useful choice. Furthermore, other desirable features and characteristics will become apparent from the subsequent detailed description and the appended claims, taken in conjunction with the accompanying drawings and this background of the disclosure.

### SUMMARY

In a first aspect of the invention, there is provided an apparatus, for determining blood pressure of a subject, comprising:
at least one processor;
at least one memory comprising computer program code;
a radio frequency (RF) wave device configured to detect pulse waveform signals from the subject; and
   wherein the at least one memory and the computer program code are configured to, with the at least one processor, determine an estimation of the blood pressure corresponding to the pulse waveform signals using a blood pressure specific transfer function (BTF) derivation model.

Preferably the RF wave device comprises a sensor and a transmitter, wherein the transmitter is configured to emit RF beams, and the sensor is configured to receive reflected RF beams from which the pulse waveform signals can be detected.

Preferably the RF wave device further comprises at least one antenna array configured to steer the RF beams.

Preferably the RF wave device is further configured to scan an arm of the subject.

Preferably a field of view of the RF wave device is equal or more than a length of the arm of the subject.

Preferably the distance from the RF wave device to the arm is substantially the same during scanning.

Preferably the RF wave device is configured to scan for pulse waveform signals at one or more measurement sites along the arm of the subject.

Preferably the RF wave device is configured to scan in successive directions.

Preferably the pulse waveform signals are separated by null-steering.

Preferably the at least one memory and the computer program code are configured to, with the at least one processor, derive a measured BTF from the pulse wave signals using the BTF derivation model, wherein the measured BTF is casually related to the blood pressure.

Preferably the at least one memory and the computer program code are configured to, with the at least one processor, infer the estimation of the blood pressure using the BTF derivation model based on the measured BTF.

Preferably the at least one memory and the computer program code are configured to, with the at least one processor, train the BTF derivation model using a blood pressure training set, wherein the blood pressure training set comprises pulse waveform signals and a corresponding true blood pressure.

Preferably the at least one memory and the computer program code are configured to, with the at least one processor, generate a generated BTF from the estimated blood pressure and compare the generated BTF with a measured BTF of the corresponding pulse waveform signals, wherein the measured BTF is derived using the BTF derivation model.

Preferably the at least one memory and the computer program code are configured to, with the at least one processor, compare the true blood pressure with the estimated blood pressure of the corresponding pulse waveform signals, wherein the estimated blood pressure is inferred using the BTF derivation model based on the measured BTF.

In a second aspect of the invention, there is provided a method, for determining blood pressure of a subject, comprising:
detecting pulse waveform signals of the subject; and
determining an estimation of the blood pressure corresponding to the pulse waveform signals of the subject using a BTF derivation model.

Preferably the method further comprises emitting RF beams and scanning to receive reflected RF beams from which the pulse waveforms signals can be detected.

Preferably the RF beams are steerable.

Preferably the method comprises scanning an arm of the subject to receive pulse waveform signals.

Preferably scanning the arm of the subject comprises maintaining substantially the same distance from the arm.

Preferably scanning the arm of the subject comprises scanning for pulse waveform signals at one or more measurement sites along the arm of the subject.

Preferably scanning the arm of the subject comprises scanning successive directions with the RF beams.

Preferably scanning the arm of the subject comprises separating the pulse waveforms via null-steering.

Preferably the method further comprises deriving a measured BTF from the pulse wave signals using the BTF derivation model, wherein the measured BTF is casually related to the blood pressure.

Preferably determining the estimated blood pressure further comprises inferring the estimated blood pressure using the BTF derivation model based on the measured BTF.

Preferably the method further comprises training the BTF derivation model by receiving a blood pressure training set, wherein the blood pressure training set comprises pulse waveform signals and the corresponding true blood pressure.

Preferably training the BTF derivation model comprises deriving a measured BTF from the pulse wave signals using the BTF derivation model.

Preferably training the BTF derivation model comprises generating a generated BTF from the estimated blood pressure and comparing the generated BTF with the measured BTF.

Preferably training the BTF derivation model comprises inferring the estimated blood pressure from the measured BTF and comparing the estimated blood pressure with the true blood pressure of the corresponding pulse waveform signal.

Any of the aforementioned features or embodiments or aspects may be combined with one or more of the other features or embodiments or aspects as described herein.

### BRIEF DESCRIPTION OF THE FIGURES

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1 shows a block diagram of an apparatus for determining a blood pressure (BP) of a subject according to an embodiment of the invention.

FIG. 2 shows a conceptual construction of an embodiment of the invention.

FIG. 3 shows a system architecture of an embodiment of the invention.

FIG. 4A depicts hemodynamics in the tube-load model of a blood vessel and FIG. 4B shows an instance of modelling BP specific transfer function (BTF) at different sites.

FIG. 5A and FIG. 5B compares beam patterns for two neighbouring arm sites of an angular separation smaller than the width of the main beam. FIG. 5A shows the main beams and FIG. 5B shows the null point beams.

FIG. 6A-6D shows examples of pulse waveforms obtained from two subjects, Subject one and Subject two, under two scenarios. FIG. 6A shows Subject one wearing a jacket. FIG. 6B shows Subject two wearing a jacket. FIG. 6C shows Subject one wearing a T-shirt. FIG. 6D shows Subject two wearing a T-shirt.

FIG. 6E-6F shows comparisons between the measured BP inferred via BTF and the true BP. FIG. 6E shows Subject one's BP comparison and FIG. 6F shows Subject two's BP comparison.

FIG. 7 shows an example neural pipeline of the BP estimator of the apparatus with the BTF-constrained deeply-recursive convolutional network (DRCN) structure.

FIG. 8 shows an example flowchart of a method for determining a BP of a subject according to an embodiment of the invention.

FIG. 9 shows an example flowchart of the deep learning (DL) training framework based on CycleGAN.

FIG. 10 shows a flow chart of a method of training the BTF derivation model.

FIG. 11A-11C shows comparisons of the measured pulse transit time (PTT) with the true PTT given different numbers of transmitter-receiver antenna pairs. FIG. 11A shows the comparison with four antenna pairs, FIG. 11B shows the comparison with six antenna pairs, and FIG. 11C shows the comparison with eight antenna pairs.

FIG. 12A-12C shows the performance of the apparatus and method to unseen subjects and scenarios in a graph. FIG. 12A shows the results of the leave-one-subject-out experiment, FIG. 12B shows the results of the leave-one-scenario-out experiment, and FIG. 12C shows the results of the leave-one-domain-out experiment.

FIG. 13A shows a table comparing the results from using the apparatus and the method with the Association for the Advancement of Medical Instruments (AAMI) standard. FIG. 13B shows a table comparing the results from using the apparatus and the method with the Britain Hypertension Society (BHS) standard.

FIG. 13C shows a table comparing the performance of the apparatus and the method with different radar placements.

FIG. 14 shows a plot of the estimation error of the apparatus and the method with different sensing schemes.

FIG. 15A-15B shows the estimation error of null point intervals. FIG. 15A shows the estimation error for systolic BP (SBP) and FIG. 15B shows the estimation error for diastolic BP (DBP).

FIG. 16A-16B shows the estimation error of training data set sizes. FIG. 16A shows the estimation error for SBP and FIG. 16B shows the estimation error for DBP.

FIG. 17A-17B shows the estimation error of weight values. FIG. 17A shows the estimation error for SBP and FIG. 17B shows the estimation error for DBP.

FIG. 18 shows the estimation error of CycleGAN-based architecture.

The skilled person will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description.

Referring to FIG. 1, there is provided an apparatus 1 for determining a blood pressure (BP) of a subject. The apparatus 1 may comprise at least one processor 2, at least one memory 3 comprising computer program code 4, and a radio frequency (RF) wave device 5. The RF wave device 5 may be configured to detect pulse waveform signals 6 from the subject. The at least one memory 3 and the computer program code 4 may be configured to, with the at least one processor 2, determine an estimation of the BP corresponding to the pulse waveform signals 6 using a BP specific transfer function (BTF) derivation model. In one embodiment, the apparatus may be called a hBP-Fi device, the name coming from hemodynamic-blood pressure and the contactless nature of the apparatus. The apparatus may be in the form of a smartphone to enable continuous opportunistic BP monitoring.

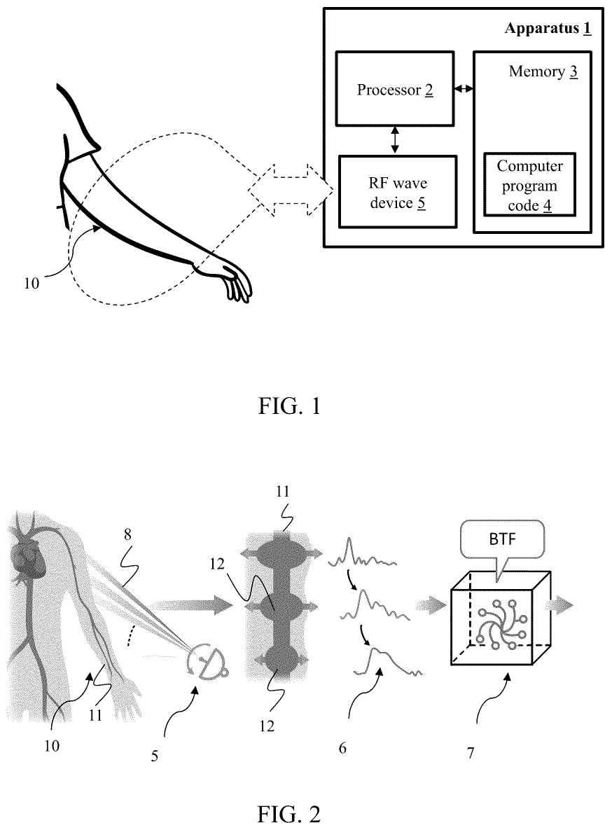

Referring to FIG. 2, hemodynamics refers to the process of how blood flows through blood vessels 11. Examples of blood vessels 11 may include arteries, capillaries, and veins. As blood flows, the blood vessels 11 undergo compliance changes indicating changes in BP. The elastic blood vessel wall pushes outwards, which then bounces back after the blood passes through. The BP indicates the radial pressure caused by the blood flow, whose continuous representation is a pulse activity. These changes result in variable pulse waveforms 6 depending on the measurement site 12. These varying pulse waveforms 6 measured at different sites allow for derivation of a BTF. The BTF describes the varying compliance along the blood vessels 11 or arteries which has a causal relationship with the BP.

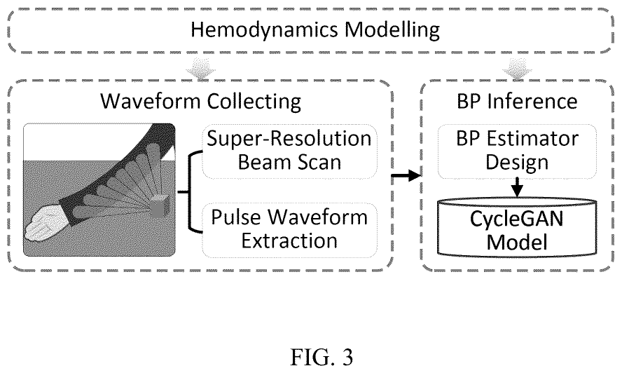

Referring to FIG. 3, the apparatus is generally composed of three components: hemodynamics modelling, pulse waveform collecting, and BP inference. Hemodynamics modelling associates the pulse activity detected by RF sensing with the implicit compliance property of the blood vessels 11. A tube-load model may be adopted to describe the hemodynamics, which leads to a novel mapping, BTF, that relates changes in the compliance to those in the pulse waveforms 6. Pulse waveform collecting comprises extracting pulse waveforms from phase changes in RF reflections to achieve adequate resolution for pulse activity sensing. BP Inference employs an explainable DL 7 pipeline, a later DL model, based on CycleGAN structure, to estimate BP in a calibration-free manner. This removes or minimises the uncertainties brought by varying subjects and scenarios.

### RF Wave Device

The RF wave device 5 may comprise a sensor and a transmitter. As shown in FIG. 2, the transmitter may be configured to emit RF beams 8. The sensor may be configured to receive reflected RF beams for detecting the pulse waveform signals 6. The RF wave device 5 may be a mmWave radar. Examples of mmWave radars include a TI AWR1843BOOST mmWave radar, or a mmwave radar on a smartphone. Other radars or RF wave devices are envisaged. The RF wave device 5 may extract pulse waveforms 6 out of the phase changes in reflected RF beams.

The RF wave device 5 may further comprise at least one antenna array configured to steer the RF beams. The antenna arrays may comprise antenna pairs comprising a transmitting antenna (Tx) and a receiving antenna (Rx). Increasing antenna pairs in the antenna array, i.e. more transmitting antenna and receiving antenna pairs, improves the angle resolution of the RF wave device 5. The antenna arrays may allow the RF beams to be steered towards certain directions. An example of antennas may be MIMO antennas but other antennas are envisaged. The RF signals transmitted and received by the RF wave device 5 may be directional, achieved by beamforming at both the transmitter and receiver sides. In an example embodiment, the RF wave device 5 may operate at 77 GHz, with three transmitter antennas and four receiver antennas, and a bandwidth of up to 4 GHz. Other numbers of antenna pairs are envisaged such as four antenna pairs, six antenna pairs, or eight antenna pairs.

The RF wave device 5 may be a continuous beam-steerable RF sensing scheme. However, during the continuous scanning process each single snapshot may not be focused on a particular site. The super-resolution scheme synthesizes all signals obtained during the scanning process to obtain a series of high-quality pulse waveforms 6, thus overcoming the problem that each snapshot may not be focused on a particular site.

The RF wave device 5 may be configured to scan an arm 10 of the subject. The apparatus 1 (FIG. 1) may steer the RF beam 8 to detect pulse waveforms 6 changes in RF reflections along the arm 10.

The field of view of the RF wave device 5 may be equal to or more than a length of the arm 10 of the subject in order for the field of view to be sufficient to cover the whole arm 10 such that the RF wave device 5 and the arm 10 does not need to move during scanning. The distance from the RF wave device 5 to the arm 10 may be substantially the same during scanning. The distance between the RF wave device 10 and the arm may be fixed. The distance between the RF wave device 5 and the arm 10 may be about 50 cm, for example. During use, the subject may be required to stretch their arms out on a surface, such as a table. The RF wave device 5 may be resting on the same surface as the arms. The subject may be required to maintain minimum or no body movement. The distance between the RF wave device 6 and the arm means the apparatus and method is contactless. In an embodiment, multiple RF wave devices 5 may be synthesized to enable room-scale contactless BP monitoring.

The RF wave device 5 may be configured to detect pulse waveform signals 6 at one or more measurement sites 12 along the arm 10 of the subject. Detecting pulse waveforms 6 from multiple sites 12 along the arm allow for hemodynamics profiling. Changes in the reflected RF beams indicate the pulse waveforms 6 along the blood vessels 11 or arteries in the arm 10 and allows the BP information to be determined through hemodynamics modelling (FIG. 3). The relationship between the BP and varying pulse waveforms 6 can be characterised as a modified tube-load model. For example, pulse waveforms 6 may be detected from two measurement sites 12.

In an example embodiment, the RF wave device 5 may be steerable to detect pulse waveforms 6 from two specific directions. The apparatus and/or method may use the pulse waveforms 6 to derive the pulse transit time (PTT), where PTT refers to the traveling time of pulse waves between two measurement sites 12. The BP may be inferred from the PTT. Pulse wave velocity (PWV) is intrinsically related to the BP variation by PWV=e/PTT where ι denotes the distance in between the two measurement sites 12. In this example, the PTT is calculated between two measurement sites 12 but the PTT, hence the BP, may be calculated for more than two measurement sites 12.

### Hemodynamics Modelling

The hemodynamics modelling of the apparatus and the method may associate the pulse activity captured by the RF wave device 5 with the implicit compliance property of the blood vessels 11. The hemodynamics modelling may be based on the tube-load model with additional accounting for blood-pressure dependent blood vessel 11 or arterial compliance or elasticity and peripheral wave reflection.

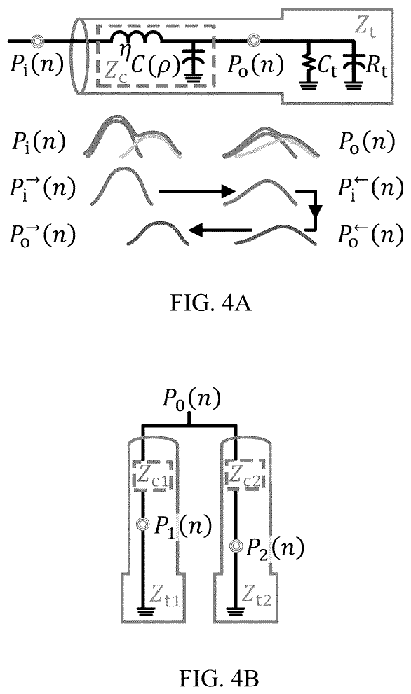

Referring to FIG. 4A, the blood vessel 11 may be modelled as a single uniform frictionless tube that has a blood-pressure related characteristic impedance of Z_{c}=\sqrt{η/C(ρ)} to support blood travel from the start to the end, where i is the constant inertance of the tube, C(ρ) is the BP-related compliance (elastic modulus) of the tube, and ρ represents the BP. According to Young's modulus, the relationship between compliance of the elastic tube and the distending BP ρ is described by the expression:

$$C ( ρ ) = {C}_{0} {e}^{- α ρ} , \qquad ( 1 )$$

where α is the blood vessel parameter and C₀ is Young's modulus for zero arterial pressure. The value of a and C₀ may be constant and subject-specific. The peripheral blood vessels 11 may be modelled as a three-element Windkessel terminal load, which has an impedance Zₜ determined by i) characteristic impedance Z_{c}, ii) peripheral resistance exerted by the arterioles Rₜ, and iii) compliance of the distal arteries Cₜ, via Zₜ=Z_{c}+Rₜ/(1+RₜCₜ).

The pulse waves at the tube inlet in time t may be denoted as Pᵢ(t) and the pulse waves at the tube outlet may be denoted as Pₒ(t). The pulse waves may consist of forward and backward waves, both traveling with delay and distortion characterised by a wave propagation function ϕ(ρ) via Pₒ^{→}(t)=e^{ϕ(ρ) ϑi}(t) and Pᵢ^{→}(t)=e^{ϕ(ρ) ϑi}Pₒ^{←}(t), where the superscripts → and ← represent the forward and backward components, respectively, and ι is the length of the tube. Moreover, the backward waves are generated by the forward ones encountering a change in impedance at the terminal load, leading to the following relation:

$${P}_{o}^{←} ( t ) = Ψ {P}_{o}^{\to } ( t ) = \frac{a s}{{s}^{2} + b s} {P}_{o}^{\to } ( t ) , \qquad ( 2 )$$

where s is the Laplace variable, α=½Z_{c}Cₜ, and b=α+1/RₜCₜ.

The at least one memory and the computer program code may be configured to, with the at least one processor, derive a measured BTF from the pulse waveform signals using the BTF derivation model. The measured BTF is casually related to the BP. Using Eqn. (2) and based on Pₒ(t) and Pᵢ(t) being the sum of forward and backward waves, the relation between Pₒ(t) and Pᵢ(t) can be modelled by a BTF Γ_{i→o} as:

$${P}_{o} ( t ) = {Γ}_{i \to 0} {P}_{i} ( t ) = \frac{1 + Ψ}{{e}^{ϕ ( ρ ) ℓ} + {e}^{- ϕ ( ρ ) ℓ} Ψ} {P}_{i} ( t ) . \qquad ( 3 )$$

Considering that the tube is frictionless, ϕ(ρ) can be reduced to \sqrt{ηC(ρ)} via the Taylor series expansion. Altogether, the BTF can be presented as:

$${Γ}_{i \to 0} = \frac{{s}^{2} + ( b + a ) s}{( {s}^{2} + b s ) {e}^{ℓ \sqrt{ηC ( ρ )}} + a s {e}^{- ℓ \sqrt{η C ( ρ )}}} , \qquad ( 4 )$$

where polynomial coefficients a, b, and constants related to C(ρ) (i.e., η, C₀, α) can be readily determined by population-based normative values or subject-specific least-squares formulation. Finally, BTF is the function of only ι and ρ. Ideally, given observed Pₒ(t) and Pᵢ(t), the BTF may be measured via Γ_{i→o}=Pₒ(t)Pᵢ⁻¹(t). Then the BP value ρ can be determined by Eqn. (4) in the least-squares sense, with prior knowledge of the precise artery length ι between the two sites. The above relation still holds when Pᵢ(t) and Pₒ(t) are superimposed by multiple pulses, which is equivalent to the parallel connection of multiple tubes with loads i.e. the multiple blood vessels in the arm.

In practice, precise measurement of ι is the major obstacle to solving BTF and thus inferring BP. However, this may be rectified by relating the BTF to the BP via optimization. For example, three pulse waveforms measured at different locations may be considered, as shown in FIG. 4B, denoted by Pₒ(t),P₁(t), and P₂(t). According to Eqn. (3), P₁(t)=Γ_{0→1}(Θ)P₀(t) and P₂(t)=Γ_{0→2}(Θ)P₀(t), where Θ [symbol] {ρ, ι_{0→1}, ι_{0→2}} defines the parameter set. Since Γ_{0→1}(Θ)⁻¹P₁(t)=P₀(t)=Γ_{0→2}⁻¹P₂(t), the parameters of Θ may be tuned by minimizing the following:

$${min}_{Θ} J ( θ ) = {min}_{Θ} {‖ {{Γ}_{0 \to 2} ( Θ )}^{- 1} {P}_{2} ( t ) - {{Γ}_{0 \to 1} ( Θ )}^{- 1} {P}_{1} ( t ) ‖}_{1} . \qquad ( 5 )$$

Several mathematical relations may be incorporated to narrow the searching space for the optimal Θ. Under the current context, RF sensing may help infer the radial range and bearing between the RF wave device 5 and the measurement sites 12, which in turn leads to estimations of ι_{0→1}, ι_{0→2}, and ι_{1→2} via trigonometry operations. More importantly, Eqn. (5) may be extended to accommodate an arbitrary number n of pulse waveforms 6 measured at different locations, further helping constrain the parameter set Θ [symbol] {ρ, ι_{0→1}, ι_{0→2}, . . . , ι_{0→n}} and potentially yielding a more accurate estimation to BP ρ.

### RF Pulse Waveform Collection

The RF wave device 5 (FIG. 1) may be configured to scan in successive directions. The steerable beam patterns may be employed in a time-division manner, enabling the RF beam 5 to scan successive directions with a microsecond-level delay to capture spatially separated pulse waveforms 6 along the arm 10.

In an embodiment of the apparatus and/or method, the subject may be placed at zero-degree bearing to ensure sufficient signal quality. In another embodiment, pulse waveforms 6 may be captured at multiple arm measurement sites 12 but require improvement of the angle-resolution.

To substantially improve the angle-resolution of the apparatus and/or the method, a uniform linear array (ULA) with N transmitter antennas (Tx) spaced by dₜₓ and M receiver antennas (Rx) spaced by dᵣₓ may be considered. To scan multiple sites, the transmitter antennas may be programmed to successively steer the RF beam towards different directions in a time-division manner, by emitting RF signals with distinct phase combinations in each time slot. During the k-th time slot, to focus the signals towards a bearing θ, the phase combination for the transmitter antennas is determined as:

$$\vec{ϕ} ( θ ) = [ {ϕ}_{1} , {ϕ}_{2} , … , {ϕ}_{N} ] = [ 0 , \frac{2 π {d}_{tx} sin θ}{λ} , … , ( N - 1 ) \frac{2 π {d}_{tx} sin θ}{λ} ] , \qquad ( 6 )$$

where λ is the signal wavelength, thus endowing directionality to the RF sensing.

However, it is highly non-trivial to separate the RF reflections from different arm measurement sites 12. The limited antennas and the short distance between the arm 10 and the RF wave device 5 forces the reflected signals from the main (or even side) beams to merge together, rendering the boundaries between neighbouring measurement sites 12 vague.

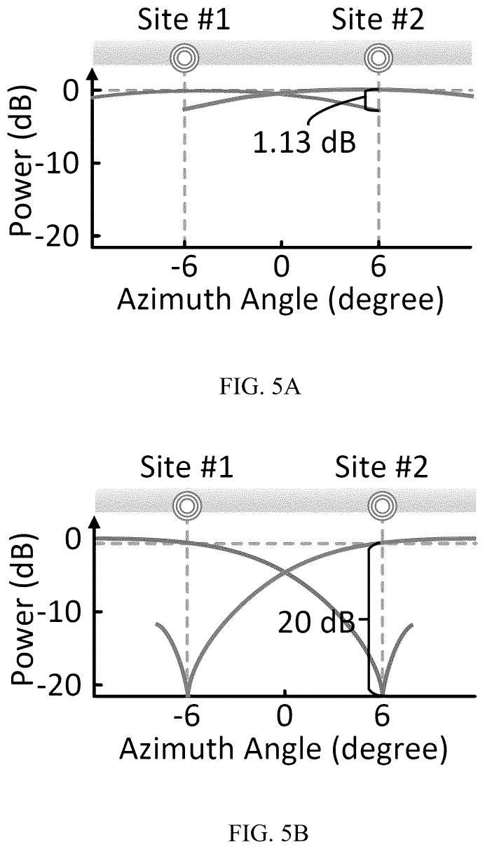

Referring to FIG. 5A, an example is shown using two main beams to demonstrate the worst case phenomenon of the merged signals for sensing pulse waveforms from two neighbouring measurement sites 12. Assuming a ULA with eight transmitter-receiver pairs with a beamwidth approximately fourteen degrees, pulse waveforms from Site #1 and Site #2 separated by twelve degrees are indistinguishable as the magnitude difference between two main beams is only 1.13 dB. Such a phenomenon makes the default RF wave device 5 beamforming scheme insufficient to obtain fine-grained pulse waveforms 6 for deriving accurate BTFs needed by Eqn. (5).

In accordance with an embodiment, the pulse waveform signals may be separated by null-steering. To magnify the difference of the RF reflections from two neighbouring measurement sites 12 with an angular separation smaller than the width of the main beam, a differential beamforming with null-steering scheme to achieve super-resolution beam scan may be used in the apparatus and/or method. Null-steering may be applied to multiple neighbouring measurement sites 12. Null-steering may be used to reject unwanted interference sources arriving from a known direction by producing a null point in the response pattern.

Referring to FIG. 5B, an example of using a ULA with eight transmitter-receiver pairs of antennas is shown. Two beams whose null points are separated by twelve degrees yield a 20 dB amplitude difference between the two main beams, rendering pulse waveforms 6 at the two measurement sites, Site #1 and Site #2, highly distinguishable. Herein, the null-steering is presented via an example of separating RF signals from two measurement sites 12 denoted by Φ₁(t) and Φ₂(t), respectively. The RF signals received in a time slot may be expressed as:

$${y}_{p} ( t ) = v ( {θ}_{1} ) {Φ}_{1} ( t ) + v ( {θ}_{2} ) {Φ}_{2} ( t ) , \qquad ( 7 )$$

where v(θ)=[1, e^{jα(θ)}, . . . , e^{j(M-1)α(θ)}] is the receiver direction vector with

$$α ( θ ) = \frac{2 π {d}_{rx} sin θ}{λ} .$$

By assigning differential weights to the received signals, the following may be formulated:

$${s}_{1} ( t ) = {w}_{1}^{H} {y}_{p} ( t ) = {[ 1 , {e}^{j α ( {θ}_{1} ) - ϑ} , … , {e}^{j ( M - 1 ) α ( {θ}_{1} ) - ϑ} ]}^{T} {y}_{p} ( t ) \qquad ( 8 )$$

$${s}_{2} ( t ) = {w}_{2}^{H} {y}_{p} ( t ) = {[ 1 , {e}^{j α ( {θ}_{1} ) + ϑ} , … , {e}^{j ( M - 1 ) α ( {θ}_{1} ) + ϑ} ]}^{T} {y}_{p} ( t ) ,$$

where ≢=π/180 is a small constant and θ₁ indicates the null point. Associating Eqn. (7)-(8):

$${s}_{1} ( t ) = {a}_{11} {Φ}_{1} ( t ) + {a}_{12} {Φ}_{2} ( t )$$

$${s}_{2} ( t ) = {a}_{21} {Φ}_{1} ( t ) + {a}_{22} {Φ}_{2} ( t )$$

$$Where$$

$$Δθ = 2 π {d}_{rx} ( sin {θ}_{2} - sin {θ}_{1} ) / λ$$

$$and$$

$$\{ {a}_{11} , {a}_{12} , {a}_{21} , {a}_{22} \} = \{ {\sum }_{m = 0}^{M - 1} {e}^{j ϑ m} , {\sum }_{m = 0}^{M - 1} {e}^{j ( Δθ + ϑ ) m} , {\sum }_{m = 0}^{M - 1} {e}^{- j ϑ m} , {\sum }_{m = 0}^{M - 1} {e}^{j ( Δθ - ϑ ) m} \}$$

According to the Euler's formula, the complex amplitude α₁₁ can be rewritten via

$${a}_{11} = {e}^{j \frac{M - 1}{2} ϑ} ( {e}^{- j \frac{M - 1}{2} ϑ} + {e}^{- j \frac{M - 3}{2} ϑ} … + {e}^{j \frac{M - 3}{2} ϑ} + {e}^{j \frac{M - 1}{2} ϑ} ) = {e}^{j \frac{M - 1}{2} ϑ} ( 2 cos \frac{( M - 1 ) ϑ}{2} + 2 cos \frac{( M - 3 ) ϑ}{2} + … ) \qquad ( 9 )$$

Applying the same derivation method, the remaining complex amplitudes can be expressed by the following:

$${a}_{12} = {e}^{- j \frac{M - 1}{2} ( Δθ + ϑ )} ( 2 cos ( \frac{( Δθ + ϑ ) ( M - 1 )}{2} ) + 2 cos ( \frac{( Δθ + ϑ ) ( M - 3 )}{2} ) + … ) ′ \qquad ( 10 )$$

$${a}_{21} = {e}^{- j \frac{M - 1}{2} ϑ} ( 2 cos ( \frac{( M - 1 ) ϑ}{2} ) + 2 cos ( \frac{( M - 3 ) ϑ}{2} ) + … ) ,$$

$${a}_{22} = {e}^{- j \frac{M - 1}{2} ( Δθ - ϑ )} ( 2 cos ( \frac{( Δθ + ϑ ) ( M - 1 )}{2} ) + 2 cos ( \frac{( Δθ + ϑ ) ( M - 3 )}{2} ) + … ) .$$

Since α₁₁ and α₂₁ have the same amplitude but only differ in phase, Φ₂(t) can be extracted by calibrating the phase difference and subtracting the calibrated signals:

$$\begin{matrix}δ s ( t ) = {s}_{2} ( t ) {e}^{j \frac{M - 1}{2} ϑ} - {s}_{1} {e}^{- j \frac{M - 1}{2} ϑ} \\ = 4 sin \frac{( M - 1 ) Δθ}{2} sin \frac{( M - 1 ) ϑ}{2} + 4 sin ( \frac{( M - 3 ) Δθ}{2} ) \\ sin ( \frac{( M - 1 ) ϑ}{2} ) + … )\end{matrix} \qquad ( 11 )$$

Moreover, Φ₁(t) may be readily obtained by replacing the null point in Eqn. (8) from θ₁ to θ₂.

### Pulse Waveform Extraction

In an embodiment of the apparatus and/or the method, the RF wave device 5 may be configured to adopt a FMCW (frequency modulated continuous wave) waveform consisting of a sine wave whose frequency increases linearly with time. The pulse induced skin vibrations may lead to minor distance variations that modulate the reflected FMCW signals, affecting their phase over time. The receiver antennas may collect FMCW reflections from the arm 10 to derive the minor distance variations based on intermediate frequency (IF) signals, i.e., differential signal between transmitter and receiver chirps, for each transmitter-receiver antenna pair independently. The IF signals may then be processed by range fast Fourier transform (FFT) to determine arm distance to the RF wave device by finding the peaks in the distance bin. This may be followed by the differential beamforming with null-steering to further separate the data from the different arm measurement sites 12. After processing multiple chirps, the signal reflected from each measurement site 12 may be combined to form a complex radar data tensor X(d, q, v), where d, q, and v indicate the distance bin, chirps, and measurement sites, respectively. Leveraging the real and imaginary components of X(d, q, v), the phase variations A<p incurred by pulse activities may be readily obtained.

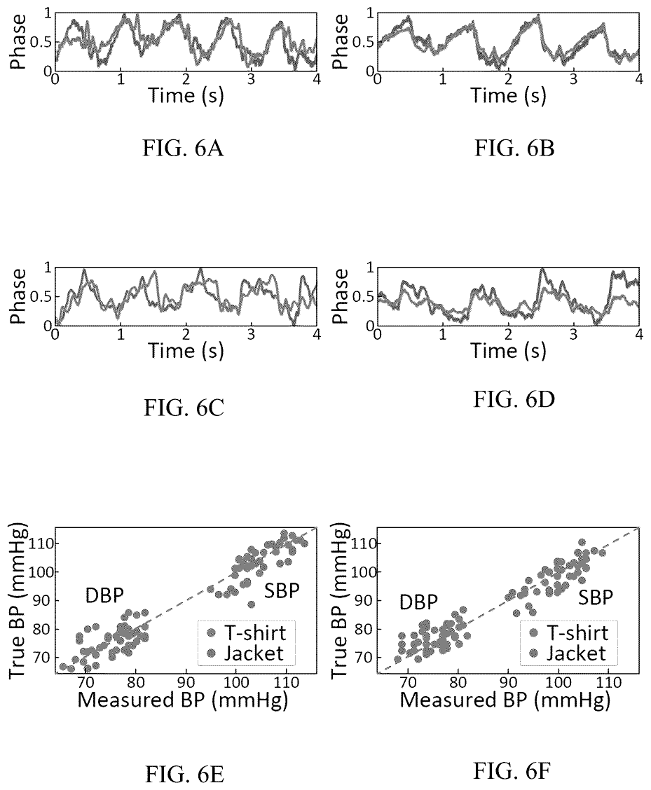

FIG. 6A-6D show examples of pulse waveforms 6 obtained from two measurement sites 12 that are separated by an angle of twelve degrees, where the lines represent the different measurement sites 12. It may be observed that pulse waveforms 6 from the two measurement sites 12 exhibit clear distinctions. Based on the collected pulse waveforms 6, the BP may be measured by searching for the optimal parameter set, as previously described.

Referring to FIG. 6E-6F, as a pilot study, fifty pairs of measured BPs, obtained by using the apparatus and method, were compared with the true BPs, obtained by an arm-cuff monitor. The results show a strong correlation between the measured BPs and true BPs, confirming the feasibility of using the BTF derivation model for BP inference. The differences in BP estimation accuracy across subjects and scenarios shows cross-subject and cross-scenario methods, for which a data-driven approach may be preferred.

### BP Inference

In an embodiment, the apparatus 1 may comprise a data capture adapter, connected to the RF wave device 5 to acquire real-time data and stream the data to the processor 2. For example, a TI DCA 1000EVM data capture adapter may be used. During data collection, the frame sampling rate may be set to 200 frames/second for example. The data processing pipeline of the processor 2 may be implemented in MATLAB R2019b and/or Python 3.8. A CycleGAN pipeline may be built upon TensorFlow 2.2. The captured FMCW reflections may be processed to obtain pulse waveforms 6, then a sliding window of 512 samples shifted by 64 samples may be applied to infer BP.

The at least one memory 3 and the computer program code 4 may be configured to, with the at least one processor 2 (FIG. 1), infer the estimation of the BP using the BTF derivation model based on the measured BTF. The combination of the mathematical model and DL may be effective in various tasks, and are typically cascaded together. First, a rough mathematical model is built to initialize the results, then DL is used to refine the models. Alternatively, DL may be used to mine the features as prior information for building an accurate mathematical model. However, both solutions separate the DL training from the mathematical model resolving, entailing manually tedious parameter adjustments while obtaining only shallow feature priors. To overcome these issues, in an embodiment of the apparatus and/or the method, the BTF derivation model may be integrated with DL by constraining the DL structure and loss based on the BTF derivation model rendering both network structure and optimization space explainable.

In an embodiment of the apparatus and/or method, the processor 2 may comprise a BP estimator built upon a deeply-recursive convolutional network (DRCN) configured to optimize the BTF derivation model parameters, which is followed by long short-term memory (LSTM) modules to obtain the BP estimates. Specifically, BTFs are first measured from the time series of varying pulse waveforms 6 via Γ_{i→o}=Pₒ(t)Pᵢ⁻¹(t).

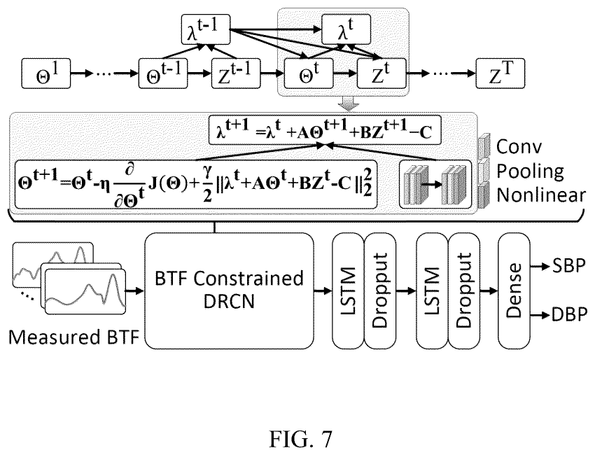

Referring to FIG. 7, the BP estimator takes the measured BTFs as input, then it outputs the optimization of Eqn. (5) into sub-problems based on an alternating direction method of multipliers as follows:

$${Θ}^{t + 1} = {Θ}^{t} - η \frac{\partial }{\partial {Θ}^{t}} J ( Θ ) + \frac{γ}{2} {‖ {λ}^{t} + A {Θ}^{t} + {BZ}^{t} - C ‖}_{2}^{2} \qquad ( 12 )$$

$${Z}^{t + 1} = DRCN ( {Θ}^{t + 1} , {λ}^{t} )$$

$${λ}^{t + 1} = {λ}^{t} + A {Θ}^{t + 1} + {BZ}^{t + 1} - C$$

which is subject to AΘ+BZ=C, and where λ is the Lagrange multiplier, γ is the penalty parameter. Each iteration is unfolded into one sub-network in recursion, which forms multicascaded sub-networks sharing the same network parameters. The BTF-constrained DRCN is the core of combining the BTF derivation model with DL, which automates the iterative optimization and variable splitting (commonly used in model-driven optimization) through DL, making the proposed DL framework explainable. The follow-up LSTM modules take the latent BP information from the optimized BTF model to finally infer systolic blood pressure (SBP) and diastolic blood pressure (DBP) through dense neuron layers.

The loss function for estimation commonly adopts the mean square error (MSE) to minimize the difference between the network output and the ground truth values. This difference potentially causes biased BP estimates due to potential DL overfitting. As the apparatus and the method is based on hemodynamics, the error of the BTF optimization is taken into account to establish the BTF-constrained loss function:

$$( {E}_{p} ) = {‖ J ( Θ ) ‖}_{2}^{2} + {‖ A Θ + BZ - C ‖}_{F}^{2} + {‖ ρ - \tilde{ρ} ‖}_{2}^{2} , \qquad ( 13 )$$

where ∥·∥_{F} represents the Frobenius norm, ρ={ρₛ, ρ_{d}} and \tilde{ρ}={\tilde{ρ}ₛ, \tilde{ρ}_{d}} denote the ground truth values and estimated values of SBP and DBP, respectively. This loss function takes the BTF-constrained parameter optimization as a regularization term, rendering the optimization space explainable.

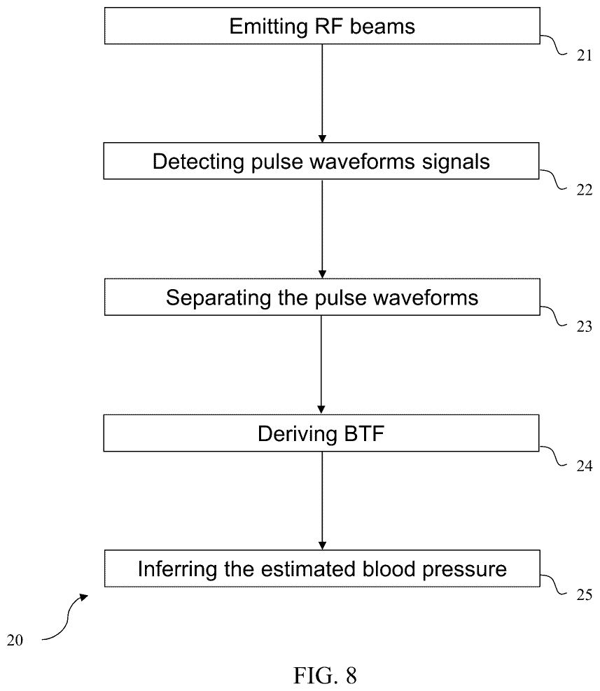

FIG. 8 shows a flowchart 20 of an example method for determining the BP of a subject and may comprise any of the embodiments and details described above. Step 21 involves emitting RF beams 8 towards a subject. The RF beams 8 are emitted by a RF wave device 5 as described above. The RF beams 8 may be steerable to allow scanning from multiple sites 12. During this continuous scanning process each single snapshot may not be focused on a particular site. However, the super-resolution scheme synthesizes all signals obtained during the scanning process to obtain a series of high-quality pulse waveforms 6, thus overcoming the problem.

Step 22 involves scanning to detect pulse waveform signals. The pulse waveform signals may be detected from the reflected RF beams 8. The reflected RF beams 8 may be received by the RF wave device 5. Optionally, the RF wave device 5 may comprise a sensor for receiving the reflected RF beams. All signals obtained during the scanning process may be synthesized to obtain a series of high-quality pulse waveforms 6.

In an example embodiment, step 22 involves scanning an arm 10 of the subject to detect pulse waveform signals 6. Substantially the same distance between the arm and the RF device may be maintained during scanning. Step 22 may also involve scanning to detect pulse waveform signals 6 at one or more measurement sites 12 along the arm 10 of the subject. The changes to the pulse waveforms from multiple sites may allow for hemodynamic profiling to determine the BP as described above.

In an example embodiment, step 22 involves scanning in successive directions with the RF beams. Scanning in successive directions may involve scanning with a microsecond-level delay to capture spatially separated pulse waveforms 6.

Step 23 involves separating the received pulse waveforms 6. This may be done utilizing null-steering in the emission of RF beams at step 21 to enhance pulse waveform separation. Step 23 may further involve magnifying the difference of the RF reflections from two neighbouring measurement sites 12 with an angular separation smaller than the width of the main RF beam. Unwanted interference sources received from a known direction may also be rejected by producing a null point in the response pattern.

Step 24 involves deriving a measured BTF from the pulse wave signals using the BTF derivation model. The BTF derivation model may be integrated with DL by constraining the DL structure and loss based on the BTF derivation model. The measured BTF is casually related to the BP.

Step 25 involves determining an estimation of the BP corresponding to the pulse waveform signals of the subject using the BTF derivation model. This may involve inferring the estimated BP using the BTF derivation model based on the measured BTF.

Training the Apparatus and/or Method

The at least one memory 3 and the computer program code 4 may be configured to, with the at least one processor 2, train the BTF derivation model using a BP training set, wherein the BP training set comprises pulse waveform signals 6 and a corresponding true BP. In an embodiment, the pulse waveform signals 6 may be detected by the RF wave device 5. The true BP may be an input received from another BP measuring device. The other BP measuring device may be an arm-cuff BP monitor for example, but other BP monitoring devices are envisaged. In order to have the BP estimator of the processor 2 calibration-free, the training procedure typically requires a high volume of training data, labelled by true SBP and DBP values, under a wide range of measurement domains, or rely on transfer learning technology. However, these methods are not feasible for training the apparatus and/or method due to the limited data available and the assumption that transfer learning on data similarity between source and target domains, which may not be true for different subjects in this scenario.

In an embodiment, CycleGAN may be used to enforce the cross-domain capability to the apparatus and/or method without the need for the aforementioned conditions or assumptions, but other AI processing techniques may also be utilized. CycleGAN may be used to achieve unpaired image-to-image translation, which typically consists of two generators and two discriminators. The rationale is to respectively learn a translation and an inverse translation between two distributions, with the goal of reducing the difference between translated data and true data, hence enforcing the generators to produce plausible results without paired data from the two domains.

The at least one memory 3 and the computer program code 4 may be configured to, with the at least one processor 2, generate a generated BTF from the estimated BP and compare the generated BTF with a measured BTF of the corresponding pulse waveform signals 6 to train the BTF derivation model. The measured BTF may be derived from the pulse wave signals using the BTF derivation model. The at least one memory 3 and the computer program code 4 may be configured to, with the at least one processor 2, compare the true BP with the estimated BP of the corresponding pulse waveform signals 6 to train the BTF derivation model. The estimated BP is inferred using the BTF derivation model based on the measured BTF. The at least one memory 3 and the computer program code 4 may be configured to, with the at least one processor 2, assess the inputs i.e. the generated BTF, the measured BTF, the true BP, and the estimated BP, and determine which inputs are the true values (i.e. the measured BTF and the true BP) and which inputs are the false values (i.e. the generated BTF and the estimated BP). In another embodiment, the values defined as true or false may be reversed. For example, the at least one memory 3 and the computer program code 4 may be configured to, with the at least one processor 2, assess the inputs, and determine which inputs are the true values (i.e. the generated BTF and the estimated BP) and which inputs are the false values (i.e. the measured BTF and the true BP).

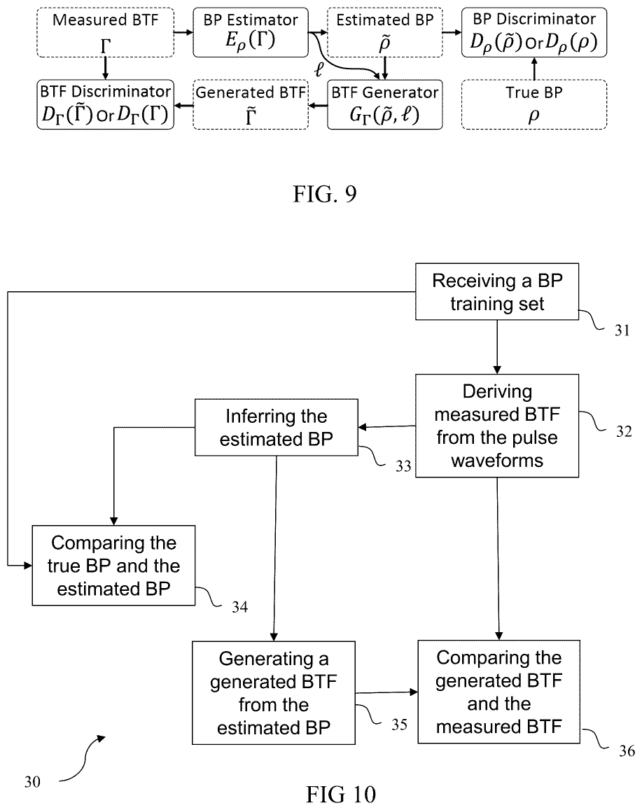

Referring to FIG. 9, the CycleGAN training architecture of an example embodiment of the apparatus and/or method is shown. The BP estimator Eρ takes the BTFs r measured from varying pulse waveforms 6 from the training set as input and estimates BP values \tilde{ρ} as output on one direction via \tilde{ρ}=Eρ (Γ). The estimated BP \tilde{ρ} and other BTF model parameters ι, derived from the measured BTF, as described above, are given as input to the BTF generator G_{Γ} to produce generated BTFs \tilde{Γ} on the reversed direction via \tilde{Γ}=G_{Γ}(\tilde{ρ}, ι). Following the BP estimator and BTF generator are two discriminators, BP discriminator D_{ρ} and BTF discriminator Dr, that complete the cyclic training process. The unpaired training forces the proposed architecture to learn stronger scenario-independent and subject-independent mapping between BTFs and BPs, making the apparatus a robust BP estimator across unseen domains with limited training data.

The BTF generator may generate domain-independent BTFs based on the estimated BPs and BTF parameters solved by the BP estimator. In one embodiment of the apparatus and/or method, an attention U-Net may be adopted as the BTF generator architecture. The encoder of the BTF generator may consist of five blocks using 64, 128, 256, and 512 filters, respectively, and the kernel size is 1×16 and the stride is two. Except for the first block, each block incorporates self-gated soft attention with layer normalization and ReLU activation after the convolutional layer to emphasize significant features conveyed through the skip connections. The decoder may adopt a similar architecture as the encoder, but with the filter quantities reversed and de-convolution layers using ReLU activation applied.

BP and BTF discriminators are built to determine whether the input comes from estimated or generated results (measured BTF and estimated BP) or true data (generated BTF and true BP) gathered in the real world from subjects. The BP discriminator takes estimated BP and true BP as inputs while the BTF discriminator takes generated BTF and measured BTF as inputs. Both discriminators utilize a common architecture consisting of four convolution layers with varying numbers of filters (64, 128, 256, and 512) for feature extraction, followed by a dense layer with softmax activation to output the probability of the input data belonging to the actual BP and/or BTF dataset. The BP discriminator D_{ρ} may be configured to receive the true BP value and the estimated BP value. The BP discriminator D_{ρ} may determine whether the values received is true, the true BP, or false, the estimated BP. In another embodiment, the values defined as true or false may be reversed, for example, the BP discriminator D_{ρ} may define the estimated BP as true, and define the true BP as false. Similarly, the BTF discriminator D_{Γ} may be configured to receive the measured BTF and the generated BTF. The BTF discriminator Dr may determine whether the values received is true, the measured BTF, or false, the generated BTF. In another embodiment, the values defined as true or false may be reversed, for example, the BTF discriminator D_{Γ} may define the generated BTF as true, and define the measured BTF as false.

The overall loss of the CycleGAN architecture is a combination of the adversarial loss and the cyclic consistency loss. Specifically, the ground truth of BP is denoted by ρ={ρₛ, ρ_{d}}. The adversarial loss of the BP estimator E_{ρ} and reliability predictor P_{ρ} is defined as follows:

$${ℒ}_{adv} ( {E}_{ρ} , {D}_{ρ} ) = [ log ( {D}_{ρ} ( ρ ) ) ] + [ log ( 1 - {D}_{ρ} ( \tilde{ρ} ) ) ] . \qquad ( 14 )$$

Similarly, the adversarial loss of the BTF generator Gr and the BTF discriminator D_{Γ} is defined as follows:

$${ℒ}_{adv} ( {G}_{Γ} , {D}_{Γ} ) = [ log ( {D}_{Γ} ( Γ ) ) ] + [ log ( 1 - {D}_{Γ} ( \tilde{Γ} ) ) ] . \qquad ( 15 )$$

The cyclic consistency loss quantifies the BP estimation loss and the BTF reconstruction loss as follows:

$${ℒ}_{cyc} ( {E}_{ρ} , {G}_{Γ} ) = ( {E}_{ρ} ( \tilde{Γ} ) ) + [ {‖ \tilde{Γ} - Γ ‖}_{2} ] . \qquad ( 16 )$$

Finally, the overall loss can be represented as:

$$ℒ = {ℒ}_{adv} ( {E}_{ρ} , {D}_{ρ} ) = {ℒ}_{adv} ( {G}_{Γ} , {D}_{Γ} ) + {ℒ}_{cyc} ( {E}_{ρ} , {G}_{Γ} ) . \qquad ( 17 )$$

The apparatus as described above may be substantially similar or the same for the method. FIG. 10 shows a flowchart 30 of an embodiment of the method for training the BTF derivation model. The steps shown in flowchart 20, may occur before or at the same time as the steps in flowchart 30.

Step 31 involves receiving a BP training set. The BP training set comprises pulse waveform signals 6 and the corresponding true BP. In an embodiment, the pulse waveform signals may be received in the same manner as step 22 from flowchart 20 as described above. The true BP may be an input received from another BP measuring device, such as an arm-cuff monitor.

Step 32 involves deriving a measured BTF from the pulse waveform signals 6. The measured BTF may be derived using the BTF derivation model. The measured BTF may be derived in the same manner as step 24 in flowchart 20 as described above.

Step 33 involves inferring the estimated BP from the measured BTF. The estimated BP may be inferred in the same manner as step 25 in flowchart 20 as described above.

Step 34 involves comparing the estimated BP, inferred in step 33, with the true BP from the training set.

Step 35 involves generating a generated BTF from the estimated BP.

Step 36 involves comparing the generated BTF, generated in step 35, with the measured BTF, derived in step 32.

### Laboratory Experiment

In an experiment, a peristaltic pump was used to emulate how blood flows through a blood vessel at different PWVs which is related to BP variation. A mmWave radar was used to perform beam-steerable sensing. Two measurement sites were chosen and the time delay between the pulse waveforms extracted from these two sites were measured as described above. The true PTT is computed as the ratio of the distance between the measurement sites and the known PWV specified by the pump.

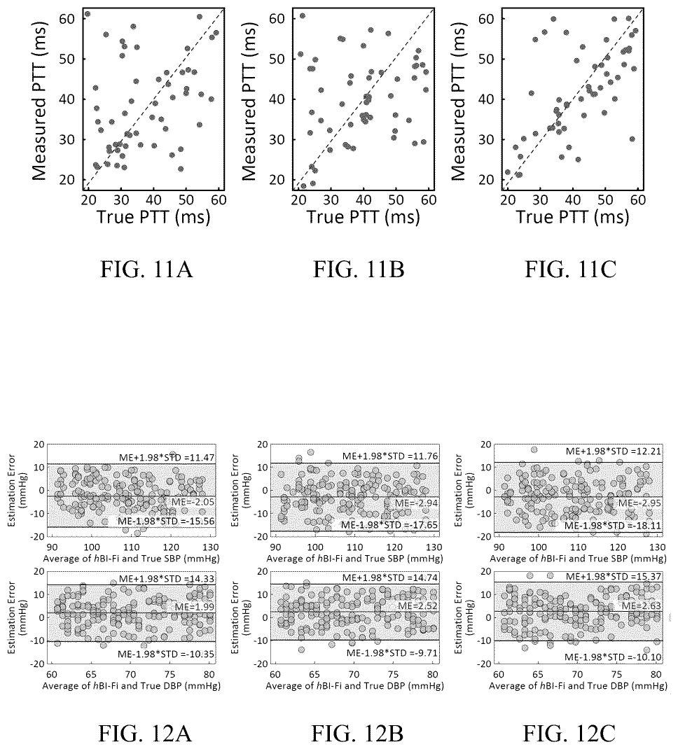

FIG. 11A-11C shows graphs comparing the measured PTTs with the true PTTs. FIG. 11A shows the comparison for four antenna pairs or four transmitter-receiver pairs. FIG. 11B shows the comparison for six antenna pairs or six transmitter-receiver pairs. FIG. 11C shows the comparison for eight antenna pairs or eight transmitter-receiver pairs. FIG. 11A-11C shows increasing antenna pairs results in the measured PTTs being closer with the true PTTs, because the angle resolution of the RF wave device is determined by the number of transmitting antenna and receiving antenna pairs. The estimation error is still present even with eight antenna pairs, indicating a deficit of a straightforward usage of beam-steerable sensing. Hence, the mathematical model that links the pulse activities and BP, as described above, is required to design a more effective beam-steerable sensing scheme for achieving super-resolution extraction of pulse waveforms.

To evaluate the apparatus and method, 35 subjects, 22 males and 13 females aged between 20 to 54 years, without known related medical conditions were recruited. The experiment setup is the standard validation procedure for BP monitoring. The subjects were asked to sit with their backs supported, legs uncrossed, and arm stretched on a tabletop. The RF wave device was placed in front of the subject with its field of view covering the whole arm area of the subject. The RF wave device scanned for pulse waveforms along the arm while the subject maintained minimal body movement. Data was collected in twelve scenarios, involving all combinations of four types of clothing worn by the subject; T-shirts, sweaters, hoodies, and winter jackets; and three types of tabletop surface materials; wood, laminate, and glass. As the relative angle and distance between the arm and the RF wave device may vary during real life use, the apparatus and method was studied with varying RF wave device placements relative to the subject. The true BP measurement was obtained using an FDA-approved arm-cuff BP monitor Omron 7127, with the arm cuff held at heart level.

Overall, approximately 252,000 heartbeat cycles were collected. The collected dataset was augmented by replicating it ten times with different random noises, providing additional resilience to overfitting.

To evaluate the apparatus and method, the mean error, ME=Σᵢ₌₁ⁿ(\tilde{ρ}ᵢ−ρᵢ)/n, and the standard deviation of the mean error STD=\sqrt{Σᵢ₌₁ⁿ(\tilde{ρ}ᵢ−ρᵢ−ME)²/n}, where \tilde{ρ} and ρ respectively denote the estimated BP and the true BP was used.

The apparatus and method for estimating BP, leveraging RF-sensed pulse waveforms, was assessed. Specifically, the apparatus and method was designed to be calibration-free and adaptable to unseen subjects and scenarios. To validate these claims, three experiments involving different combinations of subjects and scenarios were conducted.

FIG. 12A shows the results of a leave-one-subject-out experiment conducted using data from one subject for testing, and data from the remaining subjects for training using all combinations of clothing worn by the subjects and tabletop surface material. The Bland/Altman plots for the estimated SBP and DBP results are shown. More than 95% of the points are narrowly distributed within the limits of agreement, ME±1.98×STD, suggesting that the estimated BP can be an alternative to the true BP. Overall, the apparatus and method achieves a ME of −2.05 mmHg and a STD of 6.83 mmHg for estimating SBP, and a ME of 1.99 mmHg and a STD of 6.30 mmHg for estimating DBP. Such decent performance indicates that the apparatus and method may be well adapted to estimate the BP of unseen subjects.

FIG. 12B shows the results of a leave-one-scenario-out experiment, using data from one scenario (combination of clothing worn by the subject and tabletop surface material) for testing, and data from the remaining scenarios for training. The points have a narrow distribution within the ME±1.98×STD margin, demonstrating that the apparatus and the method obtains comparable BP results with the arm-cuff BP monitor. Specifically, the apparatus and method achieved a ME of −2.94 mmHg and a STD of 7.43 mmHg for estimating SBP, and a ME of 2.51 mmHg and a STD of 6.24 mmHg for estimating DBP. The low MEs and STDs shows that the apparatus and the method is scenario-independent.

FIG. 12C shows the results of a leave-one-domain-out experiment, using data from one subject in one scenario for testing, and data from the remaining subjects in other scenarios for training. The results show how the apparatus and method would perform with a new subject in a new scenario to ensure that the training and testing are mutually exclusive, i.e. how the apparatus and method would perform on subjects and scenarios unseen during training. FIG. 12C shows the results with more than 95% of the points within the limits of agreements, showing that the estimated BP and true BP can be used interchangeably. Specifically, the apparatus and method achieved a ME of −2.95 mmHg and a STD of 7.66 mmHg for estimating SBP, and a ME of 2.63 mmHg and a STD of 6.50 mmHg for estimating DBP. The apparatus and method successfully handled domain variations and enables calibration-free BP estimation for unseen subjects under unseen scenarios.

By analysing the results of the above three experiments (leave-one-subject-out, leave-one-scenario-out, and leave-one-domain-out), the apparatus and method tends to slightly underestimate the SBP and slightly overestimate the DBP, which may be due to the uneven distribution of the training data. In summary, the results confirm that the apparatus and method is capable of being an alternative solution for accurate BP monitoring and of generalizing to unseen subjects and/or scenarios in a calibration-free manner.

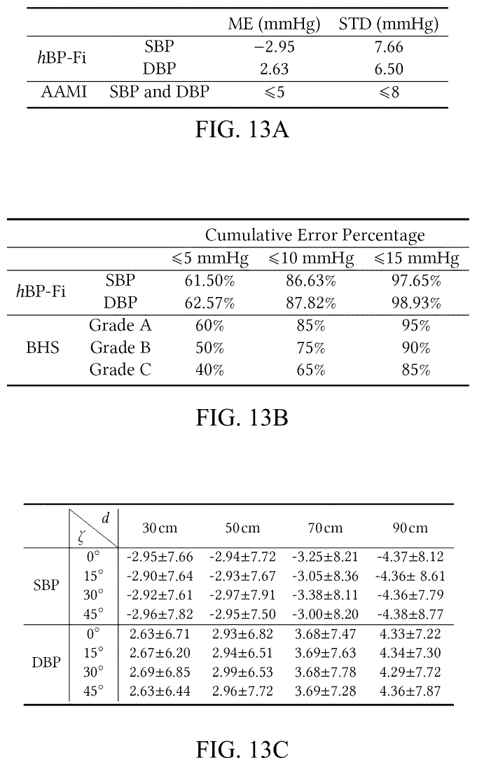

Referring to FIGS. 13A and 13B, to further validate the apparatus and the method, the results of the leave-one-domain-out experiment was compared with the acceptable range regulated by the Association for the Advancement of Medical Instruments (AAMI) and the requirements for the Britain Hypertension Society (BHS) standard. FIG. 13A compares the results of the leave-one-domain-out experiment with the acceptable range regulated by the AAMI standard. The apparatus and method satisfied the recommended error boundary defined by the AAMI. The ME for SBP and DPB are less than 5 mmHg and the STD for SBP and DBP are less than 8 mmHg. FIG. 13B compares the results of leave-one-domain-out experiment with the acceptable range regulated by the BHS standard. The apparatus and method achieved a BHS Grade A for estimating SBP and DBP. The apparatus and method are therefore constructed upon a solid physiological basis and realized by an explainable DL model to achieve a calibration-free apparatus and method for estimating BP for unseen subjects and scenarios. Its performance shows the apparatus and the method is a good alternative to the FDA-approved cuff-based BP monitor.

Referring to FIG. 13C, in real-world environments, the distance and angle between the subject's arm and the RF wave device may vary. Experiments were conducted by collecting data with distance d={30 cm, 50 cm, 70 cm, 90 cm} and angle (={0 degrees, 15 degrees, 30 degrees, 45 degrees}. FIG. 13C summarises the ME and STD quantified by the distance and the angle. Overall, the performance of the apparatus and method is stable as the angle changes. The SBP and DBP are negatively affected by the increasing sensing distance. The DBP remains within the acceptable range regulated by the FDA protocol when the distance between the subject's arm and RF wave device is within 70 cm, while the SBP falls within the acceptable range when the distance between the subject's arm and RF wave device is within 50 cm. A measure distance within 50 cm between the subject's arm and the RF wave device is reasonable in real-world use, therefore the apparatus and the method has confirmed robustness in real-world adoption.

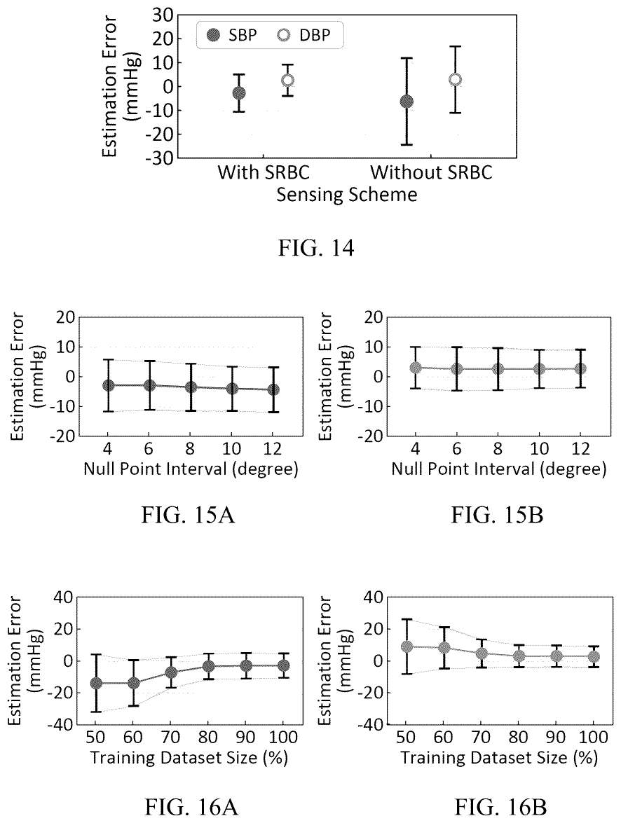

Referring to FIG. 14, an experiment was conducted to compare the super-resolution beam scan scheme (SRBC) of the apparatus and method with a traditional beamforming scheme, leaving other system components of the apparatus and method the same. The SRBC is important to obtain fine-grained BTFs and obtain accurate BP. In the case of using the traditional beamforming scheme, pulse waves derived from particular angle bins are treated as pulse waves at particular measurement sites. FIG. 14 compares the estimation error for SBP and DBP using the two sensing schemes, where the points represent the ME and the error bars indicate the STD. Without using the SRBC scheme, the estimation error between the outputs and the true BP is −5.72±17.65 mmHg for SBP and 2.89±14.01 mmHg for DBP. These estimation errors are reduced by SRBC to −2.95±7.65 mmHg for SBP and 2.63±6.50 mmHg for DBP. These results indicate that the SRBC is effective and yields more accurate BP estimates than the traditional beamforming scheme.

As described above, multiple null points evenly spaced along the arm are set, i.e., spaced at an identical angle, to separate pulse waveforms from specific arm sites. More null points may result in too little variation between pulse waveforms, making it difficult to characterise hemodynamics. However, fewer null points would result in fewer pulse waveforms being captured along the arm, which could hinder BTF optimization.

FIG. 15A shows the ME and STD for SBP estimates and FIG. 15B shows the ME and the STD for DBP estimates. To study the impact of null point distribution on the apparatus' and method's performance, data at intervals of 4 degrees, 6 degrees, 8 degrees, 10 degrees, and 12 degrees between null points were collected. These angles are all finer than the width of the main RF beam of 14 degrees. As shown by FIGS. 15A and 15B, the ME do not vary significantly from intervals four degrees to six degrees. The STD for SBP slightly decreases while the STD for DBP slightly increases. The MEs and STDs decrease after the interval reaches eight degrees and ten degrees. The MEs and STDs become relatively saturated until the interval of twelve degrees. Using the interval of ten degrees or twelve degrees achieves good performance, but twelve degrees is preferable over 10 degrees as larger intervals can save computational resources.

Referring to FIGS. 16A and 16B, a leave-one-domain-out experiment with varying amounts of training data was conducted to study the impact of training dataset size for the DL pipeline. Theoretically, a larger training data set would result in a better DL model. Data from one subject in one scenario was used for testing, while respectively using 50%, 60%, 70%, 80%, 90%, and 100% of the data from the remaining subjects in other scenarios for training. FIG. 16A shows the ME and STD for SBP estimates with varying training data sizes and FIG. 16B shows the ME and STD for DBP estimates with varying training data sizes. The ME for SBP decreased by 8.98 mmHg and the ME for DBP decreased by 3.59 mmHg as the training dataset size increased from 50% to 80%. Then the MEs come to saturation as the training dataset size increases to 90% and 100%. The STDs also follow substantially the same trend. These results indicate that the currently used training dataset is sufficient, and adding more training data only improves the BP estimation performance by a negligible margin.

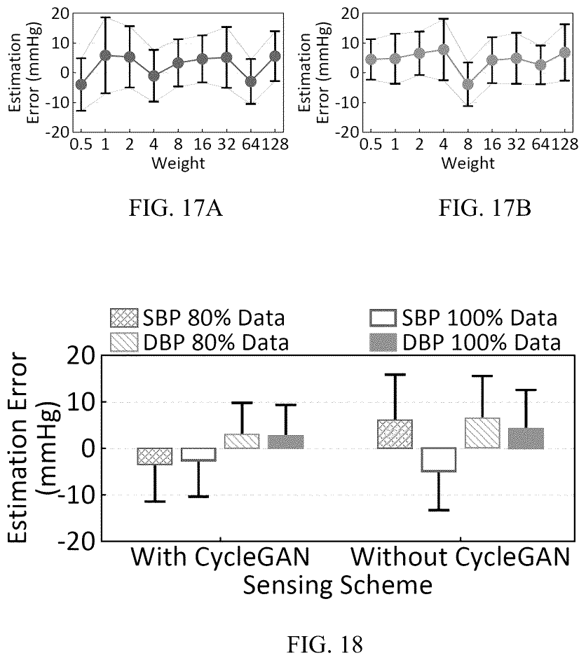

The weight of cyclic consistency loss involved in the total loss of the CycleGAN framework, i.e., β in Eqn. (17), is a crucial parameter to be tuned. In theory, larger β prevents the BP estimator from excessive hallucinations and mode collapse (both of which will cause unnecessary loss of information) but could cause unwanted artifacts. Referring to FIGS. 17A and 17B, the ME and STD for SBP and DBP with varying β values were compared to determine the optimal weight. It can be observed that MEs and STDs fluctuate as β increases but remain between −5±10 and 5±10 when β=0.5, 8, and 64, for both SBP and DBP, which is within the range required by AAMI. β=64 leads to the lowest estimation error, thus 64 is preferably used in the apparatus' and method's DL pipeline.

As described above, the CycleGAN-based architecture is calibration-free and training effective, i.e., using a reasonable amount of training data, to build an effective model that can adapt well to unknown domains. An ablation study was conducted to verify how much the CycleGAN architecture contributes to the performance of the apparatus and the method. FIG. 18 compares the MEs and STDs for SBP and DBP, with and without using the CycleGAN architecture. Using the CycleGAN architecture reduces MEs in all cases, and even without using the CycleGAN architecture, the apparatus and method still achieves results around the AAMI range, likely due to the DRCN's accurate solution of the BTF model. When using the CycleGAN-based architecture, the apparatus and method achieves decent results using a smaller amount of training data. Such results validate that the proposed CycleGAN architecture effectively handles the dependence of domains, rendering it to be calibration-free with a reasonable amount of training data.

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

As used herein the term “and/or” means “and” or “or”, or both.

While exemplary embodiments have been presented in the foregoing detailed description of the invention, it should be appreciated that a vast number of variations exist.

It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of elements and method of operation described in an exemplary embodiment without departing from the scope of the invention as set forth in the appended claims.

1. An apparatus, for determining blood pressure of a subject, comprising:
at least one processor;
at least one memory comprising computer program code;
a radio frequency (RF) wave device configured to detect pulse waveform signals from the subject; and
wherein the at least one memory and the computer program code are configured to, with the at least one processor, determine an estimation of the blood pressure corresponding to the pulse waveform signals using a blood pressure specific transfer function (BTF) derivation model.

2. The apparatus of claim 1, wherein the RF wave device comprises a sensor and a transmitter, wherein the transmitter is configured to emit RF beams, and the sensor is configured to receive reflected RF beams from which the pulse waveform signals can be detected.

3. The apparatus of claim 2, wherein the RF wave device further comprises at least one antenna array configured to steer the RF beams.

4. The apparatus of claim 1, wherein the RF wave device is further configured to scan an arm of the subject.

5. The apparatus of claim 4, wherein a field of view of the RF wave device is equal or more than a length of the arm of the subject, and/or
the distance from the RF wave device to the arm is substantially the same during scanning.

6. The apparatus of claim 1, wherein the RF wave device is configured to: scan for pulse waveform signals at one or more measurement sites along the arm of the subject, and/or
scan in successive directions.

7. The apparatus of claim 1, wherein the pulse waveform signals are separated by null-steering.

8. The apparatus of claim 1, wherein the at least one memory and the computer program code are configured to, with the at least one processor, derive a measured BTF from the pulse wave signals using the BTF derivation model,
wherein the measured BTF is casually related to the blood pressure.

9. The apparatus of claim 8, wherein the at least one memory and the computer program code are configured to, with the at least one processor, infer the estimation of the blood pressure using the BTF derivation model based on the measured BTF.

10. The apparatus of claim 1, wherein the at least one memory and the computer program code are configured to, with the at least one processor, train the BTF derivation model using a blood pressure training set, wherein the blood pressure training set comprises pulse waveform signals and a corresponding true blood pressure.

11. The apparatus of claim 10, wherein the at least one memory and the computer program code are configured to, with the at least one processor: generate a generated BTF from the estimated blood pressure and compare the generated BTF with a measured BTF of the corresponding pulse waveform signals, wherein the measured BTF is derived using the BTF derivation model, and/or compare the true blood pressure with the estimated blood pressure of the corresponding pulse waveform signals, wherein the estimated blood pressure is inferred using the BTF derivation model based on the measured BTF.

12. A method, for determining blood pressure of a subject, comprising:
detecting pulse waveform signals of the subject; and
determining an estimation of the blood pressure corresponding to the pulse waveform signals of the subject using a BTF derivation model.

13. The method of claim 12, wherein the method further comprises emitting RF beams and scanning to receive reflected RF beams from which the pulse waveforms signals can be detected.

14. The method of claim 13, wherein the RF beams are steerable.

15. The method of claim 13, wherein the method comprises scanning an arm of the subject to receive pulse waveform signals.

16. The method of claim 15, wherein scanning the arm of the subject comprises: maintaining substantially the same distance from the arm, and/or
scanning for pulse waveform signals at one or more measurement sites along the arm of the subject, and/or
scanning successive directions with the RF beams, and/or
separating the pulse waveforms via null-steering.

17. The method of claim 12, wherein the method further comprises deriving a measured BTF from the pulse wave signals using the BTF derivation model,
wherein the measured BTF is casually related to the blood pressure.

18. The method of claim 17, wherein determining the estimated blood pressure further comprises inferring the estimated blood pressure using the BTF derivation model based on the measured BTF.

19. The method of claim 12, wherein the method further comprises training the BTF derivation model by receiving a blood pressure training set, wherein the blood pressure training set comprises pulse waveform signals and the corresponding true blood pressure.

20. The method of claim 19, wherein training the BTF derivation model comprises: deriving a measured BTF from the pulse wave signals using the BTF derivation model, and/or
generating a generated BTF from the estimated blood pressure and comparing the generated BTF with the measured BTF, and/or
inferring the estimated blood pressure from the measured BTF and comparing the estimated blood pressure with the true blood pressure of the corresponding pulse waveform signal.
